(19) United States
(12) Patent Application Publication
(10) Pub. No.: US 2026/0231374 A1
(43) Pub. Date: Aug. 6, 2026

(54) Heat Sinks, Light Source Assemblies, And Apparatuses Having A Fluid Flow Diverter For Cooling LEDs Arranged Concentrically And/Or In An Elongated Direction Along A Heat Sink

(71) Applicant: Xenex Disinfection Services Inc., San Antonio, TX (US)

(72) Inventors: Deepak Jayaraj, Pittsburgh, PA (US); Nikhil Punneri Madathil, Sheboygan, WI (US)

(21) Appl. No.: 19/634,663

(22) Filed: Mar. 31, 2026

Related U.S. Application Data
(63) Continuation of application No. PCT/US2023/034361, filed on Oct. 3, 2023.

(51) Int. Cl. H05K 7/20; A61L 2/104; A61L 2/24; A61L 103/75

(52) U.S. Cl. CPC H05K 7/20409; A61L 2/104; A61L 2/24; H05K 7/20154; A61L 2103/75; A61L 2202/11; A61L 2202/14

(57) ABSTRACT

Heat sinks are disclosed which include one or more walls, a plurality of fins extending from interior surfaces of at least some of the wall/s toward an interior region, two open opposing ends at respectively opposing ends of the wall/s, and a fluid flow diverter disposed within the interior region to at least partially divert fluid flow from the interior region to areas between the plurality of fins. Light source assemblies and disinfection apparatuses are disclosed having a plurality of light emitting diodes coupled to exterior surfaces of at least some wall/s of a heat sink with such features. Additionally, the light source assemblies and/or the disinfection apparatuses are disclosed having a fluid moving device disposed adjacent to one of the two open opposing ends of the heat sink to introduce fluid into the heat sink.

### PRIORITY CLAIM

This application is a continuation application of International Application No. PCT/US2023/034361 filed Oct. 3, 2023, which designates the United States.

### BACKGROUND OF THE INVENTION

### 1. Field of the Invention

This invention generally relates to heat sinks, light source assemblies, and apparatuses, and more specifically, heat sinks, light source assemblies, and disinfection apparatuses incorporating a fluid flow diverter for cooling light emitting diodes arranged concentrically and/or in an elongated direction along a heat sink.

### 2. Description of the Related Art

The following descriptions and examples are not admitted to be prior art by virtue of their inclusion within this section.

Light emitting diodes (LEDs) offer several advantages over other light technologies, such as having lower power demands, a smaller footprint, and a longer lifespan as well as being lightweight and more eco-friendly since they do not contain harmful gasses or chemicals. LEDs, however, do have drawbacks which have hindered their adoption for some lighting applications. For instance, aside from being more costly, LEDs have a limited distribution of light, typically having a distribution angle between 60° and 130°, depending on optics of the LED. Although such directionality may be sufficient for some lighting applications, it presents a challenge for applications in which a broader distribution of light is needed. In particular, light sources for such applications generally need a greater quantity of LEDs which are strategically arranged to project light to a designated area, requiring a greater amount of power to operate the light source. As an example, a light source may include a plurality of LEDs concentrically arranged around a central region such that 360° light distribution may be achieved. Such an arrangement, however, makes thermal management of the LEDs more difficult since heat generated along the backside of the LEDs is directed toward a common central region. As is known in the art, thermal management of LEDs is important (regardless of whether the LEDs are concentrically arranged or not) since heat degrades materials making up LEDs, which in turn reduces light output, energy efficiency, and lifespan of LEDs.

Heat is a further concern for LEDs used for wavelength-specific applications since heat raises the junction temperature of LEDs, which in turn causes wavelength shifts in the output of LEDs, potentially affecting the LEDs to achieve a desired effect for a particular wavelength-specific application. For example, heat is a concern for ultraviolet (UV) LEDS since UV LEDs are generally used in applications in which a particularly peak wavelength or relatively short range of wavelengths is needed. For instance, LEDs which emit light in the UV-A and/or UV-B bands of UV radiation are often used for treating various skin conditions and concerns. Furthermore, LEDs which emit light in the UV-B and/or UV-C bands of UV radiation are often used for disinfection or sterilization applications. UV-C LEDs which are configured to emit light in the range of 260 nm to 265 nm are of particular interest since such a range is known to be optimally germicidal. The alloys used to fabricate semiconductors for current UV-B LEDS and UV-C LEDs, however, generate more heat than UV-A LEDS and visible LEDs and, thus, thermal management is even more critical for germicidal UV LEDs.

Accordingly, it would be beneficial to develop LED light source assemblies as well as components for such which improve the thermal management of the LEDs and further permit use of the LEDs in a broader array of lighting applications, such as but not limited to disinfection apparatuses.

### SUMMARY OF THE INVENTION

The following description of various embodiments of devices and methods is not to be construed in any way as limiting the subject matter of the appended claims.

An embodiment of a light source assembly includes a heat sink having one or more walls, a plurality of fins extending from interior surfaces of at least some of the one or more walls toward an interior region of the light source assembly, and two open opposing ends at respectively opposing ends of the one or more walls. The light source assembly further includes a plurality of light emitting diodes coupled to exterior surfaces of at least some of the one or more walls, a fluid moving device disposed adjacent to one of the two open opposing ends of the heat sink to introduce fluid into the heat sink, and a fluid flow diverter disposed within the interior region of the light source assembly. The fluid flow diverter is configured to at least partially divert fluid flow from the interior region to areas between the plurality of fins.

An embodiment of a heat sink includes a fluid flow diverter, one or more walls encircling the fluid flow diverter, a plurality of fins extending radially inward from interior surfaces of the one or more walls toward the fluid flow diverter, and two open opposing ends at respectively opposing ends of the one or more walls. An embodiment of a light source assembly including such a heat sink further includes a plurality of light emitting diodes coupled to exterior surfaces of at least some of the one or more walls of the heat sink.

Embodiments of disinfection apparatuses include either of the aforementioned light source assemblies with the plurality of light emitting diodes each configured to emit germicidal light. In some cases, a disinfection apparatus may include a fluid moving device disposed adjacent to one of the two open opposing ends of the heat sink to introduce fluid into the heat sink. In such configurations, the associated light source assembly may be void of a fluid moving device.

### BRIEF DESCRIPTION OF THE DRAWINGS

Other objects and advantages of the invention will become apparent upon reading the following detailed description and upon reference to the accompanying drawings in which:

FIG. 1 illustrates schematic view of an example of a light source assembly;

FIG. 2 illustrates a cross-sectional view of the light source assembly depicted in FIG. 1, taken along line A-A;

FIG. 3 illustrates a schematic view of an example of another light source assembly;

FIG. 4 illustrates a schematic view of an example of yet another light source assembly;

FIG. 5 illustrates a schematic view of an example of a disinfection apparatus; and

FIG. 6 illustrates a side perspective view of a component of the heat sink included in the light source assembly of the disinfection apparatus depicted in FIG. 5.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

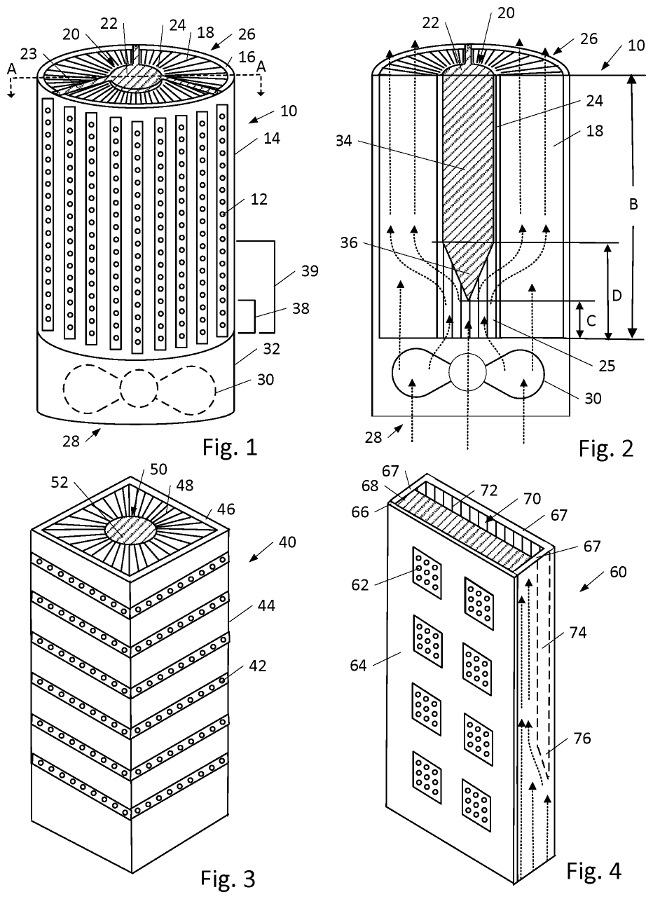

The heat sinks, light source assemblies, and apparatuses described herein incorporate a fluid flow diverter for cooling light emitting diodes (LEDs) arranged concentrically and/or in an elongated direction along a heat sink. As will be described in more detail below, the heat sinks, light source assemblies, and apparatuses may be of various configurations and may be used for various applications. Some configurations are shown and described in reference to FIGS. 1-6, but several other configurations may be considered. In particular, any of the features or combinations of features discussed below including but not limited to the features discussed in reference to FIGS. 1-6 may be applied in different configurations of a light source assembly. Furthermore, although the use of the heat sinks and light source assemblies are emphasized herein for use in disinfection apparatuses, the heat sinks and light source assemblies may be used in other apparatuses. As such, the heat sinks, light source assemblies, and apparatuses described herein are not limited to the depictions in the drawings. Furthermore, it is noted that the drawings are not necessarily drawn to scale.

Regardless of their configuration or in the application for which they are used, the light source assemblies described herein include a plurality of LEDs coupled to one or more exterior surfaces of a heat sink. The plurality of LEDs may be in any arrangement, such as one or more strips of LEDs (i.e., each strip being an array having a single row or column of LEDs), one or more two-dimensional arrays of LEDs, and/or a layout of individual LEDs dispersed across exterior surface/s of the heat sink. As described in more detail below, the plurality of LEDs of the light source assemblies described herein may, in some cases, be arranged concentrically and/or in an elongated direction along a heat sink. Alternatively stated, the plurality of LEDs may, in some embodiments, be arranged to have multiple sets of one or more LEDs each arranged a different distance from an end of the heat sink as taken along the length of the heat sink. In addition or alternatively, the plurality of LEDs of the light source assemblies described herein may be circumferentially arranged around a heat sink such that light is emitted in at least four directions from a particular point of the light source assembly (e.g., to a front side, back side, right side, and left side from a particular point of the light source assembly). In some of such cases, the LEDs may be arranged such that light is projected 360° around the light source assembly.

In any case, the plurality of LEDs may be configured to emit light in any wavelength range, depending upon the application in which the respective light source assembly is to be used. In particular, the plurality of LEDs may be configured to emit light in any wavelength range in the ultraviolet, visible, or infrared spectrum. As an example, if a light source assembly is to be used for illumination, the plurality of LEDs may be configured to emit light in the visible light spectrum. Another example of an application for the light source assemblies described herein is to be incorporated into an apparatus for disinfecting or sterilizing air and/or surfaces. In such cases, the plurality of LEDs may be configured to generate germicidal light. The term “germicidal light”, as used herein, refers to light which is capable of deactivating or killing microorganisms, particularly disease carrying and/or disease producing microorganisms (a.k.a., germs).

Ranges of light which are known to be germicidal include ultraviolet B (UVB) and ultraviolet C (UV-C) light, particularly ultraviolet light between approximately 200 nm and approximately 320 nm, and more particularly ultraviolet light at 220 nm and ultraviolet light between 260 nm and 265 nm. Another range of light which is known to be germicidal includes visible violet-blue light (also known as high-intensity narrow-spectrum (HINS) light) between approximately 400 nm and approximately 470 nm, and particularly at 405 nm. The LEDs considered for the light source assemblies and disinfection or sterilization apparatuses disclosed herein may be configured to generate any one or more of such ranges or wavelengths of germicidal light. In particular embodiments, the LEDs considered for the light source assemblies and disinfection or sterilization apparatuses disclosed herein may be configured to emit ultraviolet light at one or more wavelengths between 200 nm and 280 nm and, particularly one or more wavelengths between 260 nm and 265 nm.

In any case, the light source assemblies considered herein may, in some embodiments, include one or more barriers surrounding the LEDs on the light source assembly, specifically one or more barriers which are transparent to the light generated by the LEDs. In some cases, a light source assembly may include a single barrier surrounding the LEDs. In other embodiments, a light source assembly may include multiple barriers each surrounding a different set of one or more LEDs on the heat sink. In either case, the one or more barriers may serve to protect the LEDs from damage and/or being touched. In other embodiments, a light source assembly need not include any of such barriers, particularly if the light source assembly is arranged in an apparatus or setting in which damage or access to the LEDs is not a concern.

As shown and described in more detail below in reference to FIGS. 1-6, the heat sinks described herein may be of various configurations. For example, as described in reference to FIGS. 1-3 and 5, the heat sinks considered herein may include one or more walls encircling an interior region. In such embodiments, the heat sinks may include a single cylindrical wall or may include multiple walls conjoined along their sides to encircle a common interior region of the heat sink. In the latter scenario, the heat sinks may include any number of walls. In any case, a heat sink that includes walls/s which encircle an interior region offers a base for LEDs to be concentrically arranged along exterior surface/s of the heat sink and possibly 360° light distribution therefrom. As described in more detail below, the fluid flow diverter described herein may be particularly suitable for such configurations of heat sinks and light source assemblies since heat generated by LEDs will be transmitted to the common interior region. In other configurations, such as described in reference to FIG. 4, the heat sinks considered herein may include one or more walls which do not encircle an interior region. As described below, such a heat sink configuration may be suitable for applications in which 360° light distribution is not needed or desirable and it is not possible or desirable to dissipate heat behind the heat sink.

In any case, the heat sinks considered herein include a plurality of fins extending from interior surfaces of at least some of the one or more walls of the heat sink, and in some cases, from interior surfaces of each of the one or more walls of the heat sink. Alternatively stated, the heat sinks considered herein include a plurality of fins extending from sides of its wall/s which oppose the sides to which LEDs are arranged or will be arranged. The plurality of fins are longitudinally aligned with lengths of the one or more walls of the heat sink such that fluid introduced into an end of the heat sink may flow between the plurality of fins along the length of the heat sink. As discussed in more detail below, the plurality of fins extend toward an interior region of the light source assembly and, in some cases, an interior region of the heat sink, and a fluid flow diverter occupies at least a majority portion of the interior region (i.e., the space in the light source or heat sink extending past the plurality of fins).

In general, the plurality of fins, the interior region, and the fluid flow diverter are dimensionally configured to optimize the performance of the heat sink such that turbulence of the fluid is minimized and cooling can be achieved closer to interior surfaces of the wall/s of the heat sink (i.e., closer to the backside of the LEDs). More specifically, the surface area of the fins, the size of the interior region, and the size and shape of the fluid flow diverter are optimized in relation to the velocity of the cooling fluid to be used in the heat sink, which may be optimized in relation to the size of the heat sink and the density of the LEDs on the heat sink. In any case, the wall/s and plurality of fins of the heat sink may be of any material of relatively high thermal conductivity, examples of which may include but are not limited to aluminum and copper. Copper is generally more expensive and heavier than aluminum, but has higher thermal conductivity and better corrosion resistance than aluminum. The parameters of the light source assembly and/or the apparatus in which it will be arranged may need to be taken into consideration when selecting the material for the heat sink.

In addition to having a plurality of fins extending from interior surfaces of at least some of its one or more walls, the heat sinks considered herein include two open opposing ends at respectively opposing ends of the wall/s. In some cases, a heat sink considered herein may be prism-shaped, with its multiple walls serving as faces of the prism and its open ends serving as bases of the prism. As set forth in more detail below, the heat sinks and light source assemblies described herein may be elongated in that the length of the heat sinks between its open ends may be longer than the depth of the heat sink. The fluid flow diverter described herein may be particularly suitable for elongated heat sinks and light source assemblies and, more specifically, to include configurations by which to reduce the variation of junction temperatures of the LEDs in the elongated direction of the heat sink as explained in more detail below.

In addition to having a heat sink and a plurality of LEDs, the light source assemblies and/or apparatuses described herein include a fluid moving device adjacent to one of the two open opposing ends of the heat sink to introduce a fluid into the heat sink. The fluid may be a gas, such as but not limited to air, or a liquid. As such, the term “fluid” as used herein refers to a gas or liquid. In cases in which the fluid is a liquid or a gas other than air, the respective light source assembly and/or apparatus may include a container of the fluid and/or may be configured to be coupled to a source of the fluid. In any case, the fluid moving device may be any device configured to cause the fluid to flow, such as but not limited to a fan or a pump.

It is noted that the fluid moving device may be part of the light source assembly, but in other cases, the fluid moving device may not be part of the light source assembly. In the latter of such cases, an apparatus configured to incorporate the light source assembly may include a fluid moving device specifically arranged in the apparatus to be adjacent to one of the two opposing ends of the heat sink of the light source assembly when the light source assembly is incorporated in the apparatus. In yet other embodiments, a light source assembly may be provided as a kit with a fluid moving device separate from a light source having a plurality of LEDs attached to exterior surface/s of a heat sink and further having a compartment to receive the fluid moving device adjacent to one end of the heat sink. In any case, the areal dimension of the fluid moving device may, in some embodiments, be substantially similar (i.e., +/−15%) to the areal dimension of the space at the end of the heat sink comprising the plurality of fins and interior region of the heat sink and, in some cases, the fluid moving device may be centered with the interior region. In other cases, however, the areal dimension of the fluid moving device may be substantially smaller or larger (i.e., < or >15%) than the areal dimension of the space at the end of the heat sink comprising the plurality of fins and interior region of the heat sink.

Further to the aforementioned components, the heat sinks and/or the light source assemblies described herein include a fluid flow diverter configured to at least partially divert fluid flow introduced into the interior region of the heat sink to areas between the plurality of fins. In general, the fluid flow diverter includes a shaft extending a majority length of the heat sink and arranged in proximity and, in some cases attached, to some of the cantilevered ends of the plurality of fins such that fluid introduced into the interior region of the heat sink is blocked by the shaft and diverted to areas between the plurality of fins. Such a diversion of the fluid better cools the junction temperatures of the plurality of LEDs arranged along the heat sink since the fluid is brought closer to the LEDs and the plurality of fins dissipating heat therefrom.

In some cases, the fluid flow diverter may include a column portion and a tapered end portion, where the tapered end portion is arranged closer to the end of the heat sink to which a fluid moving device is adjacently arranged or is to be adjacently arranged (i.e., the end of the heat sink at which a fluid will be introduced into the heat sink). The tapered end portion serves to gradually divert the fluid flow to areas between the plurality of fins and reduce turbulence in the fluid flow as compared to embodiments in which the fluid flow diverter does not include such a tapered end portion. In some cases, the fluid flow diverter may have a cylindroconical shape as shown and described in reference to FIG. 2, but other shapes having a column portion and a tapered end portion may be considered. In other embodiments, a fluid flow diverter need not include a tapered end portion.

In some cases, the fluid flow diverter may include the same material as the heat sink. In other embodiments, the fluid flow diverter may include a different material than the heat sink. In some of such cases, the fluid flow diverter may include a material of relatively high thermal conductivity, such as any metal. In general, having a fluid flow diverter made of a relatively high thermal conductivity may be advantageous for transferring heat away from the plurality of fins to better cool the LEDs. In yet other cases, the fluid flow diverter may include a thermally resistant material, such as characteristic of many polymer materials. Such materials generally weigh less than metals and, thus, may be advantageous for light source assemblies or apparatuses in which weight is a concern.

In some cases, the fluid flow diverter may be configured to reduce the variation of junction temperatures of the LEDs attached to exterior surfaces of the heat sink, particularly to reduce the variation in the elongated direction of the heat sink. In particular, the junction temperature of LEDs arranged along an elongated direction of a heat sink of a conventional light source assembly may generally increase in the downstream direction of the cooling fluid flowing through the heat sink since the heat dissipated from each LED the cooling fluid passes by will be carried through the heat sink. As set forth in more detail below, a number of different fluid flow diverter configurations for reducing the variation of junction temperatures of LEDs in the elongated direction of the heat sink may be considered for the light source assemblies described herein and, in some cases, combined to achieve a desired amount of thermal management. The specifics of the possible configurations of fluid flow diverters are described in more detail below in reference to FIGS. 1-4, but the possible configurations are not specific to the configuration of the heat sinks shown in those figures. In particular, any one or more of the configurations may be employed in any configuration of a heat sink or a light source assembly described herein or an apparatus including such.

One configuration which may be used to reduce the variation of junction temperatures of LEDs in the elongated direction of a heat sink is for one end of the fluid flow diverter to be disposed inward from an end of the heat sink to which a fluid moving device is adjacently arranged or is to be adjacently arranged. A fluid flow diverter with such an inset end may generally delay the diversion of fluid from the interior region of the heat sink to areas between the plurality of fins and, consequently, the cooling fluid introduced into the interior region of the heat sink may be used to better cool LEDs arranged further downstream. In particular, it is postulated that the heat generated by the LEDs near the end of the heat sink to which a fluid moving device is adjacently arranged may be sufficiently dissipated by the fins extending from the heat sink in such a region and the fluid introduced into such fins alone will be cool enough to transfer such heat along the heat sink. Accordingly, the temperature of the cooling fluid introduced into the interior region at such a position in the heat sink may not rise as quickly as compared to embodiments in which the fluid is diverted between the fins at such a position in the heat sink. As a consequence, the cooling fluid introduced into such a region of the heat sink may be used to better reduce the junction temperatures of LEDs successively arranged in the elongated direction of the heat sink (i.e., upon the cooling fluid being diverted between the plurality of fins further downstream) and further reduce the variation of junction temperatures of LEDs along the heat sink.

In some cases, the end of the fluid flow diverter may be disposed downstream from a set of LEDs arranged closest to an end of the heat sink to which a fluid moving device is adjacently arranged or is to be adjacently arranged. In general, the set of LEDs may constitute between approximately 5% and approximately 25% of the LEDs of the light source assembly and, in some cases, may constitute between approximately 10% and approximately 20% of the LEDs of the light source assembly. In some scenarios, the set of one more LEDs may be a matrix of LEDs disposed on a common printed circuit board. In additional or alternative embodiments, the position of the end of the fluid flow diverter within the heat sink may be expressed relative to a length of the heat sink. For instance, in some cases, the end of the fluid flow diverter may be disposed inward from the end of the heat sink by a distance which is less than approximately 30% of a length of the heat sink (i.e., the dimension of the heat sink extending between its two opposing open ends) and, in some embodiments, less than approximately 15% of the length of the heat sink or less than approximately 10% of the length of the heat sink.

Another configuration which may be additionally or alternatively used to reduce the variation of junction temperatures of LEDs in the elongated direction of a heat sink is specific to the configuration of the fluid flow diverter having a column portion and a tapered end portion (a configuration which is described above for gradually diverting the fluid flow to areas between the plurality of fins). In particular, a fluid flow diverter having a tapered end portion and column portion may be configured such that the interface between the two portions is disposed downstream from the end of the heat sink to which a fluid moving device is adjacently arranged or is to be adjacently arranged. The effect and advantage of a fluid flow diverter with such a configuration is similar to having an end of the fluid flow diverter disposed inward from an end of the heat sink as described above, specifically that the diversion of fluid within the heat sink may be delayed. As a consequence, the cooling fluid introduced into the interior region of the heat sink may be used to better reduce the junction temperatures of LEDs successively arranged in the elongated direction of the heat sink and further reduce the variation of junction temperatures of LEDs along the heat sink.

In some cases, a fluid flow diverter may be configured such that the interface between its tapered end portion and its column portion is disposed downstream from at least a set of LEDs (such as but not limited to a matrix of LEDs disposed on a common printed circuit board) arranged closest to the end of the heat sink to which a fluid moving device is adjacently arranged or is to be adjacently arranged. In general, the set of LEDs may constitute between approximately 20% and approximately 60% of the LEDs of the light source assembly and, in some cases, may constitute between approximately 30% and approximately 50% of the LEDs of the light source assembly. In addition or alternatively, a fluid flow diverter may be configured such that an interface between its tapered end portion and its column portion is disposed inward from the end of the heat sink to which a fluid moving device is adjacently arranged or is to be adjacently arranged by a distance between approximately 5% and approximately 50% of a length of the heat sink (i.e., a dimension of the heat sink extending between its two opposing open ends) and, in some embodiments, between approximately 10% and approximately 30% of a length of the heat sink.

Regardless of the arrangement and/or material of the fluid flow diverter, the fluid flow diverter may, in some cases, be attached to the ends of one or more of the plurality of fins such as described for the example light source assembly depicted in FIG. 4. In such embodiments, the fluid flow diverter is an undetachable portion of the heat sink. In other cases, the cantilevered ends of the plurality of fins may not be attached to the fluid flow diverter such as described for the example light source assembly depicted in FIGS. 1 and 2.

Alternatively stated, the plurality of fins and the fluid flow diverter as well as the heat sink as a whole may be dimensionally configured such that a gap exists between the cantilevered ends of the plurality of fins and the fluid flow diverter when the diverter is placed adjacent the plurality of fins. In such embodiments, the fluid flow diverter may include support beams to attach or rest upon portions of the wall/s at one end of the heat sink such that the fluid flow diverter may be suspended adjacent the cantilevered ends of the plurality of fins. In some of such cases, the fluid flow diverter may be detachable from the heat sink, but in other cases, the fluid flow diverter may be permanently secured to the wall/s of the heat sink.

In cases in which the fluid flow diverter is detachable from the heat sink, a light source assembly may be provided as a kit with the fluid flow diverter separate from a heat sink having a plurality of LEDs attached to its exterior surface/s and an interior region dimensionally configured to receive the fluid flow diverter. Such a kit may, in some cases, also include a separate fluid moving device and the light source comprising the heat sink and LEDs may include a compartment for receiving the fluid moving device adjacent to one end of the heat sink. In other embodiments, the light source of the kit may include the fluid moving device arranged in the compartment. In yet other cases, the kit may not include a fluid moving device at all.

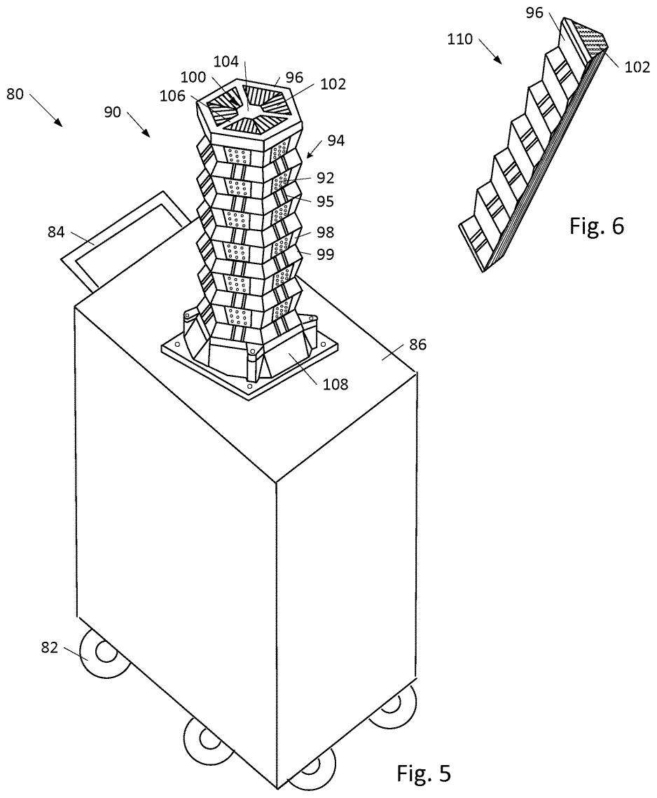

As noted above, applications in which light distribution greater than 130° is needed or desired generally require a greater quantity of LEDs and, thus, a greater amount of power to operate the light source. Some apparatuses, however, may be limited in the amount of power available for the light source due to power requirements of other components in the apparatus. An example of such an apparatus may be an area/room disinfection apparatus as described in more detail below in reference to FIG. 5, but other apparatuses may have limited power too. In view of such power constraints, it may be advantageous to limit the size of a light source assembly having LEDs and, specifically, a light source assembly having LEDs arranged to distribute light greater than 130° and up to 360° around the apparatus such that the number of LEDs along the heat sink may be minimized. For example, in some cases, the exterior surface area of the heat sinks described herein and, specifically the exterior surface of the one or more walls of the heat sinks, may be less than approximately 25 square inches and in some cases, less than approximately 15 square inches. In addition or alternatively, the interior regions of the heat sinks described herein to which the plurality of fins extend and which the fluid flow diverter chiefly occupies may be less than approximately 200 cubic inches and, in some cases, less than approximately 150 cubic inches. Heat sinks with larger interior regions and/or larger exterior surfaces, however, may be considered for some applications of light sources and, thus, the mention of such size specifications should not necessarily restrict the scope of the heat sinks, light source assemblies and apparatuses considered herein.

Turning to the drawings, examples of light source assemblies having a plurality of LEDs coupled to exterior surfaces of heat sinks and further having a fluid flow diverter disposed within the interior regions of the light source assemblies are shown in FIGS. 1-5. As noted above, the light source assemblies and heat sinks considered herein are not limited to the depictions in the drawings. For instance, any of the various configurations of features or combination of features described above for the light source assemblies and heat sinks considered herein may be applied to the light source assemblies shown in FIGS. 1-5 (i.e., to further characterize the feature/s shown in the drawings, to replace the corresponding feature/s shown in the drawings, or as additive feature/s). As such, the description of aforementioned possible features for the light source assemblies and heat sinks considered herein are referenced for each of the light source assemblies and heat sinks shown in FIGS. 1-5, but may not be reiterated for the sake of brevity. Furthermore, although the light source assemblies and heat sinks depicted in FIGS. 1-5 are shown with their elongate dimension as vertical and are further discussed with respect to the flow of fluid therein as upward, the light source assemblies and heat sinks considered herein are not necessarily limited to having such orientations for use. In particular, it is emphasized that any of the light source assemblies and heat sinks considered herein (including but not limited to those depicted in FIGS. 1-5) may be oriented with their elongate dimension as horizontal or at an angle between horizontal and vertical and/or may be orientated to induce a downward flow of fluid therein.

Light source assembly 10 is shown in FIG. 1 having a plurality of LEDs 12 coupled to exterior surfaces of heat sink 14 and, more specifically, to exterior surfaces of cylindrical wall 16. In alternative configurations, heat sink 14 may include multiple walls conjoined along their sides to encircle a common interior region of the heat sink (i.e., heat sink may include multiple contiguous walls around an interior region of the heat sink). In such cases, plurality of LEDs 12 may be coupled to exterior surfaces of some of the multiple walls and, in some cases, each of the multiple walls. In any case, as shown in FIG. 1, plurality of LEDs 12 may, in some embodiments, include one or more strips of LEDs longitudinally aligned with the length of heat sink 14. The number, length, and spacing of such strips may vary depending on the design specifications of the light source assembly and, thus, are not limited to the depiction in FIG. 1. In alternative configurations, plurality of LEDs 12 may include one or more strips of LEDs horizontally arranged around heat sink 14. In other cases, plurality of LEDs 12 may include one or more strips of LEDs arranged at an angle between 0° and 90° relative to the ends of heat sink 14. In yet other configurations, plurality of LEDs 12 may include one or more two-dimensional arrays of LEDs and/or a layout of individual LEDs dispersed across exterior surface/s of the heat sink. In any case, plurality of LEDs 12 may, in some embodiments, be arranged around heat sink 14 such that 360° light distribution may be emitted from light source assembly 10. In other cases, plurality of LEDs 12 may be arranged along the exterior surfaces of heat sink 14 such that that less than 360° light distribution may be emitted from light source assembly 10.

In some cases, plurality of LEDs 12 may be arranged in an elongated direction along heat sink 14. In particular, plurality of LEDs 12 may include multiple sets of one or more LEDs each arranged a different distance from an end of the heat sink. For instance, each LED of the LED strips depicted in FIG. 1 may constitute a set of LEDs with LEDs of the other strips at their same level and, in effect, define a set of LEDs arranged a set distance from an end of heat sink 14. Each of the successively arranged LEDs of the multiple strips may constitute a different set of LEDs at a different distance from the end of heat sink 14. In addition, plurality of LEDs 12 may be concentrically arranged around heat sink 14 such that light is emitted in at least four directions from a particular point of light source assembly 10 (e.g., to a front side, back side, right side, and left side from a particular point of the light source assembly). In some of such cases, the LEDs may be arranged such that light is projected 360° around light source assembly 10.

In any case, plurality of LEDs 12 may be configured to emit light in any wavelength range, depending upon the application in which the light source assembly 10 is to be used. In particular, plurality of LEDs 12 may be configured to emit light in any wavelength range in the ultraviolet, visible, or infrared spectrum. As set forth in more detail below, the light source assemblies described herein may, in some embodiments, be incorporated into an apparatus for disinfecting or sterilizing air and/or surfaces. In such cases, plurality of LEDs 12 may be configured to generate germicidal light. Ranges of light which are known to be germicidal include ultraviolet B (UVB) and ultraviolet C (UV-C) light, particularly ultraviolet light between approximately 200 nm and approximately 320 nm, and more particularly ultraviolet light at 220 nm and ultraviolet light between 260 nm and 265 nm. Another range of light which is known to be germicidal includes visible violet-blue light (also known as high-intensity narrow-spectrum (HINS) light) between approximately 400 nm and approximately 470 nm, and particularly at 405 nm. In particular embodiments, plurality of LEDs 12 may be configured to emit ultraviolet light at one or more wavelengths between 200 nm and 280 nm.

In addition to cylindrical wall 16, heat sink 14 is shown in FIG. 1 as having plurality of fins 18 concentrically extending from interior surfaces of cylindrical wall 16 toward interior region 20. The number, width, length, and spacing of fins 18 as well as the size of interior region 20 may vary depending on the design specifications of the light source assembly and, thus, are not limited to the depiction in FIG. 1. For instance, the surface area of plurality of fins 18 and the spacings therebetween may be dimensionally configured in relation to the velocity of a cooling fluid to be used in the heat sink such that turbulence of the fluid flowing through the fins is minimized. In addition, interior region 20, and particularly fluid flow diverter 22 disposed therein, may be configured to optimize the performance of the heat sink such that interior surfaces of cylindrical wall 16 may be better cooled (i.e., to dissipate heat more efficiently from the backside of plurality of LEDs 12).

As shown in the cross-sectional view of light source assembly 10 in FIG. 2 taken along line A-A in FIG. 1, plurality of fins 18 are longitudinally aligned with lengths of cylindrical wall 16. As further shown in FIG. 2, interior region 20 extends between cantilevered ends of plurality of fins 18 and, thus, includes fluid flow diverter 22 disposed therein as well as spacing 24 between fluid flow diverter 22 and the cantilevered ends of plurality of fins 18. Spacing 24 in interior region 20 between fluid flow diverter 22 and the cantilevered ends of plurality of fins 18 may be advantageous for configurations in which it may be beneficial for fluid flow diverter 22 to be detachable from heat sink 14. In particular, configuring fluid flow diverter 22 to be detachable from heat sink 14 may be advantageous for simplifying the process of manufacturing heat sink 14 or light source assembly 10. In alternative configurations, fluid flow diverter 22 may be coupled to some of plurality of fins 18 and, thus, spacing 24 may be omitted in part or entirely from light house assembly 10.

In any case, interior region 10 is shown in FIGS. 1 and 2 as being central in heat sink 14, which may be beneficial for embodiments in which plurality of LEDs 12 are circumferentially arranged around heat sink 14, such as to provide 360° or nearly 360° light distribution. In particular, having interior region 10 central to heat sink 14 in such scenarios may be beneficial for providing more uniform dissipation of the heat generated by plurality LEDs 12 in such an arrangement. In other cases, interior region 20 need not be central in heat sink 14. For example, uniform heat dissipation may not be particularly important for some applications in which light source assembly 10 may be used (such as but not limited to lighting applications which are not wavelength specific and/or applications in which the intensity of the light needs to be maintained above a certain threshold). In some cases, having interior region 20 off-center in heat sink 14 may be beneficial. For example, in scenarios in which most or all of plurality of LEDs 12 are arranged along a particular side of heat sink 14, it may be advantageous for the heat sink to include longer fins extending from the interior surfaces of such a side of the heat sink to facilitate greater heat dissipation from such an area of the heat sink.

As further shown in FIGS. 1 and 2, heat sink 14 includes open opposing ends 26 and 28 at respectively opposing ends of cylindrical wall 16 and further includes fan 30 disposed adjacent end 28 to introduce a gas, such as but not limited to air, into the heat sink. Fan 30 is shown by dashed lines in FIG. 1 to illustrate the fan is disposed within the interior of housing 32. In some cases, housing 32 may be an extension of cylindrical wall 16 (or any other configuration of walls making up heat sink 14) and, thus, end 28 of heat sink 14 may be at the end of housing 32 as shown in FIGS. 1 and 2. In other cases, housing 32 may be a separate material from cylindrical wall 16 and, in some embodiments, not substantially conductive. In such configurations, end 28 of heat sink 14 may be at the interface of cylindrical wall 16 and housing 32. In any case, open opposing ends 26 and 28 are fluidly connected with spaces between plurality of fins 18 such that a fluid introduced at end 28 by fan 30 is routed in the spaces between the plurality of fins along the length of the heat sink and discharged at end 26. As shown in FIG. 2 and described in more detail below, fluid flow diverter 22 may be configured such that void space 25 exists in interior region 20 of heat sink 14 adjacent to plurality of fins 18 near end 28. In such cases, fluid introduced at end 28 by fan 30 is routed into voided space 25 and then is diverted downstream from end 28 to spaces between plurality of fins 18 by fluid flow diverter 22 as is shown by the curved dotted arrowed lines in FIG. 2.

As further shown in FIGS. 1 and 2, the areal dimension of fan 30 may, in some embodiments, be substantially similar (i.e., +/−15%) to the areal dimension of end 28 comprising plurality of fins 18 and interior region 20 and, in some cases, fan 30 may be centered with the interior region. In other cases, the areal dimension of fan 30 may be substantially smaller or larger (i.e., < or >15%) than the areal dimension of end 28 comprising plurality of fins 18 and interior region 20. In any case, fan 30 may be replaced by a different fluid moving device, such as but not limited to a pump, in other configurations, particularly if a liquid cooling fluid is to be introduced into heat sink 14. In yet other embodiments, light source assembly 10 may be void of a fluid moving device and, in such cases, may be used in a device or apparatus having a fluid moving device to which end 28 of heat sink 14 may be adjacently arranged. In any case, fluid flow diverter 22 is shown in FIGS. 1 and 2 having support beams 23 to attach or rest upon portions of cylindrical wall 16 and possibly the top portions of some of plurality of fins 18 at end 26 of heat sink 14, allowing fluid flow diverter 22 to be suspended within interior region 20. In some cases, support beams 23 may be permanently fastened to cylindrical wall 16 and/or the top portions of some of plurality of fins 18 and, thus, fluid flow diverter 22 may be undetachable from heat sink 14. In other embodiments, support beams 23 may be removably fastened to cylindrical wall 16 and/or the top portions of some of plurality of fins 18 or may not be fastened to heat sink 14 and, thus, fluid flow diverter 22 may be detachable from heat sink 14.

As shown in FIG. 2, fluid flow diverter 22 includes column portion 34 and tapered end portion 36, with the tapered end portion arranged closer to end 28 of heat sink 14 to which fan 30 is adjacently arranged. Although fluid flow diverter 22 is shown as cylindroconical, column portion 34 and tapered end portion 36 may be shaped differently. For example, column portion 34 may be a prism shape, the sides of tapered end portion 36 may be concave, and/or the end portion of tapered end portion 36 may not be pointed. In any case, tapered end portion 36 serves to gradually divert the fluid flow to areas between the plurality of fins and reduce turbulence in the fluid flow through heat sink 14. Despite such an advantage, fluid flow diverter 22 may not include tapered end portion 36 in some embodiments. In such cases, column portion 34 may extend further down interior region 20 to any of the elevations described below for tapered end portion 36. In any case, column portion 34 may extend more than a majority of the length of the heat sink.

As shown in FIG. 2, the tip of tapered end portion 36 is disposed inward from end 28 of heat sink 14 by distance C, which is generally less than approximately 15% of length B of heat sink 14 extending between ends 26 and 28, but may be longer in other configurations of light source assemblies. Alternatively, the position of the tip of tapered end portion 36 may be expressed as being downstream from LED set 38 shown in FIG. 1, which includes the two lowermost LEDs on each of the LED strips disposed on heat sink 14 and constitutes less than 15% of the LEDs in light source assembly 10. It is noted that the tip of tapered end portion 36 may be disposed downstream from a LED set having more or less LEDs (and consequently having a higher or lower percentage of the LEDs from light source assembly 10) depending on the layout and makeup of plurality of LEDs 12. In any case, having tip of tapered end portion 36 disposed inward from end 28 of heat sink 14 may generally delay the diversion of fluid from void space 25 to areas between plurality of fins 18. As a consequence, the cooling fluid introduced into void space 25 may be used to better cool LEDs arranged further downstream (in the direction toward end 26 of heat sink 14) and may reduce the variation of junction temperatures of plurality of LEDs 12 in the elongated direction of heat sink 14 as discussed in more detail above. Despite such an advantage, the tip of tapered end portion 36 may alternatively be level with end 28 in some cases or may even extend past end 28.

Regardless of the position of the tip of tapered end portion 36, the length of tapered end portion 36 may aid in reducing the variation of junction temperatures of plurality of LEDs 12 in the elongated direction of heat sink 14. In particular, the length of tapered end portion 36 may be configured such that its interface with column portion 34 is disposed downstream from end 28 of heat sink 14. As shown in FIG. 2, the interface between column portion 34 and tapered end portion 36 may be disposed inward from end 28 of heat sink 14 by distance D, which may generally be between approximately 35% and approximately 45% of length B of heat sink 14, but may be shorter or longer in other configurations of light source assembly 10. Alternatively, the position of the interface between column portion 34 and tapered end portion 36 may be expressed as being downstream from LED set 39 shown in FIG. 1, which constitutes less than 40% of the LEDs in light source assembly 10. It is noted that the interface between column portion 34 and tapered end portion 36 may be disposed downstream from a LED set having more or less LEDs (and consequently having a higher or lower percentage of the LEDs from light source assembly 10) depending on the layout and makeup of plurality of LEDs 12.

Turning to FIG. 3, light source assembly 40 is shown having features of different configuration than light source assembly 10 of FIGS. 1 and 2. Although not shown, the inside of light source assembly 40 may include any of the components and associated configurations described above for light source assembly 10. Descriptions of such components and configurations are not reiterated for the sake of brevity. Furthermore, it is noted that the features discussed below in reference to light source assembly 40 are not necessarily mutually exclusive. The features are depicted together to highlight variations of different features described above which may be considered for the heat sinks, light source assemblies and apparatuses considered herein, but in no way serve to limit the scope of the heat sinks, light source assemblies and apparatuses to that what is depicted in FIG. 3.

As shown in FIG. 3, light source assembly 40 has plurality of LEDs 42 coupled to exterior surfaces of heat sink 44 and, more specifically, to exterior surfaces of walls 46 of heat sink 44. Plurality of LEDs 42 are shown having multiple strips of LEDs horizontally arranged along exterior surfaces of two walls of heat sink 44. In some cases, the multiple strips may be circumferentially arranged around heat sink 44, but in other embodiments the multiple strips may be arranged around less than the entirety of heat sink 44. In any case, the number, size, and spacing of the strips may vary depending on the design specifications of the light source assembly and, thus, are not limited to the depiction in FIG. 3. In other embodiments, plurality of LEDs 42 may alternatively have a single strip of LEDs horizontally arranged along exterior surfaces of one or more walls of heat sink 44. In addition or alternatively, plurality of LEDs 42 may include one or more strips of LEDs longitudinally arranged along the length of heat sink 44 such as described for plurality of LEDs 12 in FIG. 1. In other cases, plurality of LEDs 42 may include one or more strips of LEDs arranged at an angle between 0° and 90° relative to ends of heat sink 44. In yet other configurations, plurality of LEDs 42 may include one or more two-dimensional arrays of LEDs and/or a layout of individual LEDs dispersed across exterior surface/s of the heat sink. In any case and regardless of their orientation and arrangement, plurality of LEDs 42 may, in some embodiments, be arranged around heat sink 44 such that that 360° light distribution may be emitted from light source assembly 40. In other cases, plurality of LEDs 42 may be arranged along the exterior surfaces of heat sink 44 such that that less than 360° light distribution may be emitted from light source assembly 40.

Heat sink 44 differs from heat 14 of light source assembly 10 shown in FIG. 1 in that heat sink 44 includes four walls 46 conjoined along their sides to encircle interior region 50 of the heat sink and, to specifically have a shape of a rectangular prism. Plurality of LEDs 42 may be coupled to exterior surfaces of some of walls 46 and, in some cases, each of the walls of heat sink 44. In alternative configurations heat sink may include a different number of walls, including a single cylindrical wall such as shown for light source assembly 10 in FIG. 1 or including a different plurality of walls to have a different prism shape. In any case, heat sink 44 includes plurality of fins 48 extending from its wall/s toward interior region 50 and further includes fluid flow diverter 52 occupying the interior region. Fluid flow diverter 52 differs from fluid flow diverter 22 of light source assembly 10 shown in FIG. 1 in that fluid flow diverter 52 is coupled to at least some of plurality of fins 48 and furthermore does not include support beams to attach to or rest upon walls 46 of heat sink 44. With such a configuration, fluid flow diverter 52 may be an undetachable portion of heat sink 44. In some cases, fluid flow diverter 52 may be cast with some of the plurality of fins 48 coupled thereto, making the fluid flow diverter a permanent feature of heat sink 44 and having the same material as heat sink 44. In any case, fluid flow diverter 52 is not limited to having a cylindrical shape, but alternatively may include a prism shape and, in some cases, may be the same prism shape as walls 46. In other embodiments, fluid flow diverter 52 may be of a prism shape that is different than the prism shape of walls 46. In any case, fluid flow diverter 52 may include any of the configurations described above for fluid flow diverter 22 in reference to FIG. 2.

Turning to FIG. 4, light source assembly 60 is shown having features of different configuration than light source assemblies 10 and 40 of FIGS. 1-3. Although not shown, light source assembly 60 may include a fluid moving device within the lowermost portion of the light source assembly, specifically below taper end portion 76 of fluid flow diverter 72 to introduce a fluid into the light source assembly. In other cases, light source assembly 60 may not include a fluid moving device, but its bottom may be arranged in proximity to one for operation. In any case, as discussed in more detail below, light source assembly 60 is configured to divert a cooling fluid introduced at the bottom of light source assembly 60 to the spaces between plurality of fins 68 and out the top end of the fins to dissipate the heat generated by plurality of LEDs 62.

One of the most distinguishing characteristics of light source assembly 60 relative to light source assemblies 10 and 40 is that heat sink 64 of light source assembly 60 does not encircle an interior region. Rather, heat sink 64 includes single conductive wall 66 coupled with thermally resistant walls 67 of light source assembly 60 to encircle an interior region 70 of the light source assembly. Such a configuration for a light source assembly may be suitable for applications in which 360° light distribution is not needed or desirable and it is not possible or desirable to dissipate heat behind and/or to the side of heat sink 64. An example of such an application may include but is not limited to a device having such a light source assembly disposed within the walls of the device and heat generated from the backside of the LEDs may hinder the interior components of the device. Although heat sink 44 is specifically depicted having a single thermally conductive wall 66 coupled with three thermally resistant walls 67 along the respective two sides and backside of the light source assembly, the number of the respective walls may be different (i.e., heat sink 64 may include multiple walls and light source assembly 60 may include less than or more than three thermally resistant walls) and may, in combination, be more than four walls or may be three walls. Alternatively stated, heat sink 64 may generally include at least one conductive wall coupled with at least one thermally resistant wall of a light source assembly to encircle an interior region of the light source assembly.

In any case, light source assembly 60 has plurality of LEDs 62 coupled to exterior surfaces of thermally conductive wall 66 of heat sink 64. Plurality of LEDs 62 are shown as multiple two-dimensional arrays of LEDs, but plurality of LEDs 62 may alternatively include a single two-dimensional array of LEDs. Furthermore, the multiple two-dimensional arrays of plurality of LEDs 62 shown in FIG. 4 need not be arranged in linear columns and rows. In particular, plurality of LEDs 62 may include multiple two-dimensional arrays dispersed across thermally conductive wall in a different pattern or having no pattern. In any case, the number, size, and spacing of the two-dimensional arrays as well as the number of LEDs on each array may vary depending on the design specifications of the light source assembly and, thus, are not limited to the depiction in FIG. 4. In other embodiments, plurality of LEDs 62 may include one or more strips of LEDs arranged in any of the orientations described above for plurality of LEDs 12 and 42 of light source assemblies 10 and 40 of FIGS. 1-3. In yet other embodiments, plurality of LEDs 62 include a layout of individual LEDs dispersed across exterior surfaces of thermally conductive wall 66. In any case, since heat sink 64 does not itself encircle an interior region, the light distribution from light source assembly 60 is less than 360°.

As shown in FIG. 4, heat sink 64 includes plurality of fins 68 extending from wall 66 toward interior region 70 and further includes fluid flow diverter 72 occupying the interior region. Fluid flow diverter 72 may be spaced apart or coupled to plurality of fins 68 and, respectively, may or may not include support beams to attach to or rest upon thermally resistant walls 67. The sideways contour of fluid flow diverter 72 is shown in FIG. 4 by dashed lines on one of thermally resistant walls 67 to illustrate the fluid flow diverter is disposed within light source assembly 60. The contour of plurality of fins 68 disposed within light source assembly 60 is not shown to simplify the drawing. As is shown in FIG. 4, fluid flow diverter 72 may include main portion 74 having substantially uniform depth and tapered end portion 76. Similar to tapered end portion 36 of light source assembly 10 described in reference to FIG. 2, tapered end portion 76 may generally delay the diversion of fluid from a void space below tapered end portion 76 to areas between plurality of fins 68. As a consequence, the cooling fluid introduced into the void space may be used to better cool LEDs arranged further downstream (in the direction toward the top of light source assembly 60) and may reduce the variation of junction temperatures of plurality of LEDs 62 in the elongated direction of heat sink 64.

Turning to FIG. 5, disinfection apparatus 80 is shown including light source assembly 90 having features of different configurations than light source assemblies 10, 40 and 60 of FIGS. 1-4. Although not shown, the inside of light source assembly 90 may include any of the components and associated configurations described for light source assembly 10 that is shown and described in reference to FIG. 2. Descriptions of such components and configurations are not reiterated for the sake of brevity. Furthermore, it is noted that the features discussed below in reference to light source assembly 90 are not necessarily mutually exclusive. In addition, any of the different configurations of features of light source assembly 90 may be combined or substituted into any of the light source assemblies and heat sinks disclosed herein including but not limited to those of light source assemblies 10, 40 and 60 of FIGS. 1-4. Moreover, light source assembly 90 is not restricted for use with a disinfection apparatus, much less an area/room disinfection apparatus as emphasized for disinfection apparatus 80 below. In particular, any of the light source assemblies disclosed herein, including but not limited to light source assembly 90, may be included in any apparatus in which a lighting source is needed. In addition, any of the light source assemblies disclosed herein, including but not limited to light source assembly 90, may be configured for independent operation. In such cases, the light source assemblies may include a power source or at least connections for accessing power to operate the LEDs and fluid moving device of the light source assembly.

As noted above, disinfection apparatus 80 is emphasized as an area/room disinfection apparatus. As used herein, the term “area/room disinfection apparatus” refers to an apparatus configured to disinfect a space which is suitable for human occupancy so as to deactivate, destroy or prevent the growth of disease-carrying microorganisms in the area. As used herein, the terms “area/room disinfection apparatus”, “area/room disinfection device”, “area disinfection apparatus”, “area disinfection device”, “room disinfection apparatus”, and “room disinfection device” may be used interchangeably herein. The phrase “a space which is suitable for human occupancy” as used herein refers to a space in which an adult human being of average size may comfortably occupy for at least a period of time to eat, sleep, work, lounge, partake in an activity, or complete a task therein. In some cases, spaces suitable for human occupancy may be bounded and include a door for entering and exiting the room. In other cases, a space suitable for human occupancy may be an area with indeterminate boundaries. Examples of spaces which are suitable for human occupancy include but are not limited to single patient rooms, multiple occupancy patient rooms, bathrooms, walk-in closets, hallways, bedrooms, offices, operating rooms, patient examination rooms, waiting and/or lounging areas and nursing stations. Although disinfection apparatus 80 is described below as having one or more configurations to facilitate area/room disinfection, disinfection apparatus 80 need not be so limited. As such, disinfection apparatus 80 may include, but need not include all or any of the features described below that are generally associated with area/room disinfection devices.

In general, an area/room disinfection device includes configurations to distribute an effective amount of germicide in a spacious manner to an ambient of a room in which the device is arranged to maximize the number of surfaces and objects disinfected in the room. The device may be of any shape, size, or configuration in which to achieve such an objective. An example configuration of an area/room disinfection device which may be particularly considered for disinfection apparatus 80 is for light source assembly 90 to be arranged to distribute germicidal light approximately 360° around the assembly. Configurations and arrangements of light source assembly 90 to achieve such an objective are described in more detail below. Furthermore, as shown in FIG. 5, disinfection apparatus 80 may be void of a component upon the upper surface of support structure 86 adjacent to light source assembly 90 that is sufficient to block germicidal light emitted therefrom. As a result, germicidal light emitted from light source assembly 90 may substantially encircle the apparatus at least 1 meter from the apparatus. In additional or alternative cases, disinfection apparatus 80 may include a reflection unit above or below light source assembly 90 to redirect light emitted from the light source assembly to encircle the apparatus. Examples of reflection units which offer 360° light redirection for area/room disinfection apparatuses are disclosed in U.S. patent application Ser. No. 13/706,926 filed Dec. 6, 2012 and Ser. No. 13/708,208 filed Dec. 7, 2012 and International Patent Application No. PCT/US2014/059698 filed Oct. 8, 2014, all of which are incorporated herein by reference as if set forth fully herein.

Other features which may be additionally or alternatively included in disinfection apparatus 80 to specifically affect room/area disinfection are those that affect portability of the apparatus, such as wheels 82 and/or a handle 84. In particular, is often preferred for area/room disinfection devices to be readily portable such that they may be moved to multiple rooms of a building. Another configuration of a room disinfection device is to be automated to move through a room or area while the germicidal source is projecting germicide into an ambient of the room or area. For instance, some room disinfection devices include motorized wheels and processor-executable program instructions for activating the motorized wheels in accordance with a predetermined route and/or in response to sensors to maneuver around obstacles in the room or area while the germicidal source is emitting germicide/s. It is noted, however, disinfection apparatus 80 need not include features that affect portability of the apparatus. In particular, disinfection apparatus 80 may be dimensionally configured to be small enough to be carried by an individual or may be disposed in a fixed location in an area or room and still serve as an area/room disinfection apparatus.

In any case, other features specific to room disinfection devices which may be included in disinfection apparatus 80 include configurations for remotely starting the apparatus such that individuals need not be present in the room for operation of the device. Another component which is often included in room disinfection devices is a movement detection sensor and/or a room/area occupancy sensor, such as a motion sensor, a thermal sensor, a Doppler sensor, or a photo recognition sensor. In particular, to prevent inadvertent exposure of germicidal light, particularly UV-C light, to individuals during disinfection of a room or an area, disinfection apparatus 80 may include program instructions to inhibit or terminate activation of a power supply circuit to the light source assembly 90 upon detecting movement and/or occupancy in the area/room in which the apparatus is arranged.

Another common characteristic of room disinfection devices which disinfection apparatus 80 may be configured is to direct germicidal light to a region approximately 2 feet and approximately 4 feet from a floor of a room in which the apparatus is arranged. In particular, the region between approximately 2 feet and approximately 4 feet from a floor of a room is considered a “high touch” region of a room since objects of frequent use are generally placed in such a region. Examples of configurations for disinfection apparatus 80 which may offer such light direction include but are not limited to having light source assembly 90 disposed at an elevation between approximately 2 feet and 4 feet from a floor of the room. In addition or alternatively, disinfection apparatus 80 may, in some cases, include a reflection unit adjacent light source assembly 90 to redirect light emitted from the light source assembly to a region adjacent the disinfection apparatus and which is between approximately 2 feet and 4 feet from a floor of the room. Examples of reflection units which light redirection to a high touch region of an area/room are disclosed in U.S. patent application Ser. No. 13/706,926 filed Dec. 6, 2012 and Ser. No. 13/708,208 filed Dec. 7, 2012 and International Patent Application No. PCT/US2014/059698 filed Oct. 8, 2014, which as noted above are incorporated herein by reference as if set forth fully herein. It is noted, a reflection unit providing light redirection to a high touch region of an area/room may be the same or different from a reflection unit included in disinfection apparatus 80 that provides 360° light distribution around the disinfection apparatus as referenced above.

Another characteristic of a room disinfection device which disinfection apparatus 80 may be configured is to distribute an effective amount of germicidal light to achieve at least a 2-log reduction in bacterial contamination on surfaces within a room or area that are greater than 1 meter or even 2 or 3 meters from the disinfection apparatus. In some cases, a room disinfection apparatus having such a configuration (i.e., configured is to distribute an effective amount of germicidal light to achieve at least a 2-log reduction in bacterial contamination on surfaces within a room or area that are greater than 1 meter or even 2 or 3 meters from the disinfection apparatus) may be referred to herein as a “whole room microbial reduction device.” In some embodiments, disinfection apparatus 80 may be configured is to distribute an effective amount of germicidal light to achieve at least a 6-log reduction in bacterial contamination on surfaces within a room or area that are greater than 1 meter or even 2 or 3 meters from the disinfection apparatus.

In any case, disinfection apparatus 80 may, in some embodiments, be configured to distribute germicidal light generally uniformly (i.e., having less than a 15% variation of irradiance) in a region a set distance from the disinfection apparatus, such as but not limited to 1, 2, or 3 meters from the disinfection apparatus, during a disinfection cycle of the apparatus. Configurations of light source assembly 90 to achieve either or both objectives are described in more detail below. In some cases, disinfection apparatus 80 may include components other than the configurations of light source assembly 90 to aid in achieving a desired reduction in bacterial contamination on surfaces within a room or area and/or general irradiance uniformity at a set distance from the disinfection apparatus. For example, disinfection apparatus 80 may, in some embodiments, include an actuator coupled to light source assembly 90 and processor-executable program instructions for activating the actuator to move the light source assembly while the light source assembly is projecting germicidal light to aid in the distribution of the germicidal light in a room or area. More specifically, light source assembly 90 may be moved in vertical, horizontal, and/or diagonal directions via the actuator while the light source assembly is projecting germicidal light into an ambient of a room or area.

Yet another feature which may be included in disinfection apparatus 80 is processor executable program instructions for receiving data regarding characteristics of an enclosed space in which the disinfection apparatus is to be operated. In some cases, disinfection apparatus 80 may include one or more sensors for detecting characteristics of an area or room. In addition or alternatively, disinfection apparatus 80 may include an input device for receiving the information from a user of the apparatus or a separate device. In any case, the phrase “characteristics of an enclosed space” as used herein refers to physical attributes as well as non-physical attributes of an enclosed space. Non-physical attributes of an enclosed space include but are not necessarily limited to identifiers used to reference an enclosed space (e.g., room number and/or room name) and occupancy information regarding an enclosed space (e.g., infection information of a patient previously occupying the space or a patient scheduled to occupy the space). Physical attributes of an enclosed space include but are not necessarily limited to size and/or dimensions of the enclosed space and/or the number, size, distances, locations, reflectivity and/or identification of surfaces, objects and/or items within the enclosed space. In some cases, a physical attribute of an enclosed space may be the identification of one or more pathological organisms and, sometimes further the number or concentration of such organism/s in the enclosed space, in a particular region of the enclosed space, or on a particular surface in the enclosed space. In any case, the data received regarding the characteristics of the enclosed space in which disinfection apparatus 80 is to be operated may be utilized in a number of manners, including but not limited to recordation or reporting purposes or setting one or more operational parameters of the apparatus.

Regardless of the features disinfection apparatus 80 includes, the disinfection apparatus may generally include support structure 86 upon which light source assembly 90 is disposed. In general, support structure 86 may house and/or provide surfaces upon which to mount components to affect the operation of light source assembly 90 and disinfection apparatus 80 as a whole. The operational components may include but are not limited to circuitry, a central processing unit, a storage medium comprising program instructions to affect operation of components of the disinfection apparatus, a user interface, a room occupancy sensor and, in some cases, a power source. As shown in FIG. 5, light source assembly 90 is arranged upon and extends above the upper surface of support structure 86 such that its elongated dimension is vertically oriented. Such an arrangement of light source assembly 90 may be beneficial for distributing germicidal light 360° around disinfection apparatus 80, particularly when light source assembly 90 has LEDs arranged around its circumference as set forth below and further when disinfection apparatus 80 is void of a germicidal light blocking component upon the upper surface of support structure 86. In some cases, disinfection apparatus 80 may include a reflection unit above or below light source assembly 90 to further facilitate 360° light distribution around the disinfection apparatus.

In some embodiments, disinfection apparatus 80 may be configured such that germicidal light is distributed to a high touch region of the area or room in which the disinfection apparatus is arranged, particularly a region laterally adjacent disinfection apparatus 80 (as opposed to above disinfection apparatus) and which is between approximately 2 feet and approximately 4 feet from a floor of a room such that objects in the high touch region may be disinfected. In some cases, disinfection apparatus 80 may be configured such that germicidal light is distributed to an entire vertical span of such a region. A configuration to affect either of such objectives is having at least a portion of light source assembly 90 disposed at an elevation between approximately 2 feet and 4 feet from a floor of the room. In the configuration depicted in FIG. 5, the extension of light source assembly 90 above the upper surface of support structure 86 facilitates such objectives as well. In some cases, disinfection apparatus 80 may include a reflection unit adjacent light source assembly 90 to redirect light emitted from the light source assembly to a region laterally adjacent the disinfection apparatus and which is between approximately 2 feet and 4 feet from a floor of the room. In the configuration shown in FIG. 5 in which light source assembly 90 is arranged upon the upper surface of support structure 86 and 360° light distribution is desired, a reflector disposed above and having a width greater than light source assembly 90 may be particularly beneficial for such an objective.

As further shown in FIG. 5, the upper surface of support structure 86 may, in some cases, be at substantially the same elevation as handle 84 (i.e., within six inches of each other). Alternatively, the upper surface of support structure 86 may be at an elevation substantially higher than handle 84 (i.e., an elevation more than six inches higher than the elevation of the handle). In any case, having the upper surface of support structure 86 at the same or higher elevation than handle 84 and further having light source assembly 90 disposed upon the upper surface of support structure 86 without any germicidal blocking component arranged adjacent to it may be beneficial for minimizing the amount of light that is blocked from being distributed around the apparatus, particularly by handle 84. In some embodiments, however, the upper surface of support structure 86 may be at an elevation substantially lower than handle 84 (i.e., an elevation more than six inches lower than the elevation of the handle). In any case, handle 84 may generally be disposed approximately 36 inches above a floor of a room in order to make disinfection apparatus 80 ergonomic for pushing or pulling the unit.

Light source assembly 90 is shown in FIG. 5 having heat sink 94 having six walls 96 conjoined along their sides to encircle interior region 100 and, to specifically have a shape of a hexagonal prism. In conjunction with the size of light source assembly 90 and the number, size, and spacing of the LED arrays disposed on heat sink 94, the hexagonal shape of heat sink 94 was found, during the development of the light source assemblies disclosed herein, to provide 360° and substantially uniform light distribution (i.e., having less than a 15% variation of irradiance) in a region 1 meter away from disinfection apparatus 80. Heat sink 94, however, may be configured in other shapes and/or may include a different number of walls, including but not limited to a single cylindrical wall such as shown for light source assembly 10 in FIG. 1 or a different plurality of walls to have a different prism shape, and be configured to provide 360° and substantially uniform light distribution in a region 1 meter away from disinfection apparatus 80.

In any case, it may be advantageous to limit the size of heat sink 94 and, more specifically, the exterior surface area of heat sink 94, in order for light source assembly 90 to provide 360° light distribution and/or substantially uniform light distribution. In particular, limiting the exterior surface area of heat sink 94 facilitates a closer arrangement of LEDs around the circumference of the heat sink which may lend to 360° light distribution and/or substantially uniform light distribution. An example size restriction of the exterior surface area of the heat sink 94 and, specifically the exterior surface area of walls 96 in combination, may be less than approximately 25 square inches and, in some cases, less than approximately 15 square inches. To provide such a limited exterior surface area, the elongated dimension of heat sink 94 may, in some cases, be restricted, such as between approximately 5 inches and approximately 20 inches. Heat sinks with larger exterior surfaces and/or longer elongated dimensions, however, may be considered for light source assembly 90 and, thus, the mention of such size specifications should not necessarily restrict the scope of heat sink 94, light source assembly 90, and disinfection apparatus 80. Restricting the size of heat sink 94, however, may also be advantageous for limiting the weight of heat sink 94 since disinfection apparatus 80 is configured for portability.

In any case, one or more walls 96 of heat sink 94 may, in some embodiments, be multifaceted and, in particular cases, may have alternating downward facing panels 98 and upward facing panels 99 arranged at angles between 0° and 90° relative to a horizontal plane of the light source assembly, in effect forming an accordion pattern as shown in FIG. 5. Plurality of LEDs 92 are coupled to downward facing panels 98 and wiring 95 connecting the LEDs along the elongated direction of heat sink 94 extend between the LEDS along the intervening upward facing panels 99. During the development of disinfection apparatuses having the light source assemblies disclosed herein, the accordion pattern of panels 98 and 99 as well as the placement of plurality of LEDs 92 on downward facing panels 98 was found to be advantageous for directing germicidal light to a high touch region of the area or room adjacent the disinfection apparatus while limiting the size of the light source assembly.

In some embodiments, panels 98 having plurality of LEDs 92 coupled thereto are longer than panel 99 to accommodate more LEDs. In some specific cases, panels 98 may be approximately 20% to approximately 70% longer than panels 99 and, more specifically, between 25% and 50% longer than panels 99. In yet other cases, however, panels 98 and 99 may have approximately similar lengths (i.e., within 15% of each other) or panels 98 may have smaller lengths than panels 99. In any case, the angles to which panels 98 and 99 are slanted relative to a vertical plane of disinfection apparatus may generally be between approximately 5% and approximately 30%, but smaller or greater angles may be used. Furthermore, the number of panels 98 and 99 may be the same or different and may generally vary among different light source assemblies, depending on their design specifications. An example range for the number of panels along each of walls 96 may generally be between 3 and 10 for each of panels 98 and 99.

As noted above, light source assembly 90 may include LEDs arranged around its circumference to facilitate 360° light distribution around disinfection apparatus 80. In such cases, light source assembly 90 may include plurality of LEDs 92 coupled to exterior surfaces of each of walls 96 of heat sink 94. In other embodiments, light source assembly 90 may have plurality of LEDs 92 coupled to less than each of walls 96 of heat sink 94, particularly if 360° light distribution is not needed or desired or if 360° light distribution can be achieved without doing so. Plurality of LEDs 92 are shown having multiple two-dimensional arrays of LEDs, but plurality of LEDs 92 may alternatively include a strip of LEDs or individual LEDs dispersed across exterior surface/s of wall/s 96 of heat sink 94. In any case, the number, size, and spacing of the arrays or the individually arranged LEDs may vary depending on the design specifications of light source assembly 90 and disinfection apparatus 80.

In view of light source assembly 90 being incorporated into a disinfection apparatus, plurality of LEDs 92 are LEDs configured to generate germicidal light, particularly ultraviolet light between approximately 200 nm and approximately 320 nm and/or HINS light between approximately 400 nm and approximately 470 nm. In particular embodiments, plurality of LEDs 92 are configured to emit ultraviolet light at one or more wavelengths between 200 nm and 280 nm and, in some cases, one or more wavelengths between 260 nm and 265 nm. In any case, plurality of LEDs 92 may be configured to generate a power flux of germicidal light sufficient to achieve a 2-log reduction in bacterial contamination on surfaces within a room or area that are greater than 1 meter from the germicidal source within a given disinfection period. In cases in which UV-C light is used, a power flux of approximately 1.0 W/m² of UV-C may be generally sufficient to achieve at least 2-log reduction in bacterial contamination on surfaces within a room or area that are greater than 1 meter from the light source assembly within a given disinfection period.

As shown in FIG. 5, heat sink 94 includes plurality of fins 102 concentrically extending from interior surfaces of walls 96 toward interior region 100. The number, width, length, and spacing of fins 102 as well as the size of interior region 100 may vary depending on the design specifications of the light source assembly and, thus, are not limited to the depiction in FIG. 5. As noted above, it may be advantageous in some cases to limit the size of heat sink 94 to affect 360° light distribution, substantially uniform light distribution, and/or limit the weight of heat sink 94. Thus, in some cases, the size of interior region 100 (i.e., the interior volume of heat sink 94 extending between the cantilevered ends of plurality of fins 102 and which fluid flow diverter 104 chiefly occupies) may be limited. For example, in some cases, interior region 100 may be less than approximately 200 cubic inches and, in some cases, less than approximately 150 cubic inches. Heat sinks with larger interior regions, however, may be considered for light source assembly 90 and, thus, the mention of such size specifications should not necessarily restrict the scope of heat sink 94, light source assembly 90, and disinfection apparatus 80.

In any case, heat sink 94 may, in some embodiments, be cast as a whole, including walls 96 and plurality of fins 102. In other embodiments, it may be advantageous to fabricate heat sink 94 as multiple pieces and fasten them together for the formation of light source assembly 90. An example of heat sink piece for heat sink 94 is shown in FIG. 6. In particular, FIG. 6 illustrates heat sink piece 110 having one of walls 96 with plurality of fins 102 extending from the side opposing panels 98 and 99. Heat sink piece 110 is shown in FIG. 6 without plurality of LEDs 92 coupled to panels 98 and without wiring 95 coupled to panels 99, but illustrates groove 112 on panels 99 for receiving wiring 95. In general, plurality of LEDs 92 and wiring 95 may be attached to heat sink piece 110 before or after the heat sink piece is coupled with the other heat sink pieces of heat sink 94 to make up light source assembly 90.

Light source assembly 90 is shown in FIG. 5 including fluid flow diverter 104 occupying interior region 100 and having support beams 106 attached to or resting upon walls 96 of heat sink 94. The shape and size of fluid flow diverter 104 may vary depending on the design specifications of the light source assembly. Furthermore, fluid flow diverter 104 may not include support beams in some embodiments. In any case, although not shown, fluid flow diverter 104 may have any of the configurations described above for fluid flow diverter 22 described above in reference to FIGS. 1 and 2 for light source assembly 10, and particularly the configurations of fluid flow diverter 22 shown interior to heat sink 14 in FIG. 2. Descriptions of such components and configurations are not reiterated for the sake of brevity. In general, fluid flow diverter 104 may include a shaft extending at least a majority length of the heat sink and be arranged in proximity to cantilevered ends of plurality of fins 102 such that fluid introduced into a space adjacent the plurality of fins of the heat sink is blocked by the shaft and diverted to areas between the plurality of fins.

As further shown in FIG. 5, light source assembly 90 includes base 108 below the lowermost panel 99 of heat sink 94. Base 108 may be used to secure light source assembly 90 to the upper surface of support structure 80. In some cases, base 108 may have a fluid moving device within its interior, such as described for fan 30 of light source assembly 10 in reference to FIG. 1. In other embodiments, disinfection apparatus 80 may include a fluid moving device within support structure 86, which light source assembly 90 may be aligned and exposed to through base 108. In either case, heat sink 94 includes two open opposing ends at respectively opposing ends of walls 96 such that a fluid can be introduced into the lowermost end of the heat sink by the fluid moving device, routed through the spaces between plurality of fins 102 (including via diversion of the fluid near the lowermost end of the heat sink by fluid flow diverter 104), and discharged out the uppermost end of the heat sink.

As set forth above, light source assembly 90 is arranged upon and extends beyond the upper surface of support structure 86. Such an arrangement may be particularly advantageous for embodiments in which light source assembly 90 is the only germicidal light source in disinfection apparatus 80 which is configured to project germicidal light into an ambient of apparatus 80 (i.e., exterior to apparatus 80). It is noted, however, the disinfection apparatuses considered herein are not limited to apparatuses having a light source assembly of such an arrangement. In particular, a disinfection apparatus considered herein may, in some embodiments, have an LED light source assembly arranged partially or wholly inset to a structure of the apparatus with an opening or transparent window to allow the transmission of germicidal light emitted therefrom to be projected into an ambient of the apparatus. In addition, a disinfection apparatus considered herein may have any number of LED light source assemblies arranged along any portion of the apparatus, including the top portion, sidewall portions or the bottom of the apparatus. In yet other cases, the disinfection apparatuses considered herein need not be configured to project germicidal light into their ambient. For instance, a disinfection apparatus which is configured to draw in air, disinfect the air internal to the apparatus via one or more LED light source assemblies therein, and project the disinfected air back into the room/area in which the disinfection apparatus is arranged may be considered herein.

It will be appreciated to those skilled in the art having the benefit of this disclosure that this invention is believed to provide heat sinks, light source assemblies, and apparatuses having a fluid flow diverter for cooling LEDs arranged concentrically and/or in an elongated direction along a heat sink. Further modifications and alternative embodiments of various aspects of the invention will be apparent to those skilled in the art in view of this description. For example, although the discussion provided herein emphasizes use of such heat sinks and light source assemblies for use in an area/room disinfection apparatus, the heat sinks and light source assemblies may be used in other apparatuses. In particular, it is contemplated the features of the heat sinks and light source assemblies described herein may be incorporated in apparatuses used in a variety of other industries, particularly but not limited apparatuses used for wavelength-specific applications and/or applications in which light distribution greater than 130° is desired or needed. Moreover, the light source assemblies disclosed herein may be configured for independent operation (i.e., the light source assemblies need not be part of an apparatus to operate).

Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims. The term “approximately” as used herein refers to variations of up to +/−5% of the stated number.

What is claimed is:

1. An apparatus comprising a light source assembly and a support structure underlying the light source assembly, wherein the support structure houses components to affect operation of the light source assembly, and wherein the light source assembly comprises:
a heat sink, comprising:
one or more walls;
a plurality of fins extending from interior surfaces of at least some of the one or more walls toward an interior region of the light source assembly; and
two open opposing ends at respectively opposing ends of the one or more walls;
a plurality of light emitting diodes coupled to the exterior surfaces of at least some of the one or more walls;
a fluid moving device arranged adjacent to one of the two open opposing ends of the heat sink and configured to introduce a fluid into the heat sink in a downward direction; and
a fluid flow diverter arranged within the interior region of the light source assembly, wherein the fluid flow diverter is configured to at least partially divert fluid flow from the interior region to areas between the plurality of fins.

2. The apparatus of claim 1, wherein the one or more walls of the heat sink encircle the interior region of the light source assembly.

3. The apparatus of claim 2, wherein the one or more walls comprise a cylindrical wall multiple contiguous walls around the interior region.

4. The apparatus of claim 1, wherein an end of the fluid flow diverter is disposed inward from the end of the heat sink to which the fluid moving device is adjacently arranged.

5. The apparatus of claim 4, wherein the end of the fluid flow diverter is disposed inward from the end of the heat sink to which the fluid moving device is adjacently arranged by less than approximately 30% of a length of the heat sink extending between its two opposing open ends.

6. The apparatus of claim 1, wherein an end of the fluid flow diverter is disposed downstream from at least a first set of one or more light emitting diodes arranged closest to the end of the heat sink to which the fluid moving device is adjacently arranged.

7. The apparatus of claim 1, wherein the fluid flow diverter comprises a shaft having a main portion and a tapered end portion, wherein the tapered end portion is arranged closer to the end of the heat sink to which the fluid moving device is adjacently arranged.

8. The apparatus of claim 7, wherein an interface between the main portion and the tapered end portion is disposed downstream from at least a first set of one or more light emitting diodes arranged closest to the end of the heat sink to which the fluid moving device is adjacently arranged.

9. The apparatus of claim 7, wherein an interface between the main portion and the tapered end portion is disposed from an end of the heat sink to which the fluid moving device is adjacently arranged by a distance that is between approximately 5% and approximately 50% of a length of the heat sink extending between its two opposing open ends.

10. The apparatus of claim 1, wherein the fluid flow diverter extends a majority length of the interior region.

11. The apparatus of claim 1, wherein the plurality of light emitting diodes are each configured to emit germicidal light.

12. The apparatus of claim 11, wherein the plurality of light emitting diodes are each configured to emit ultraviolet light at one or more wavelengths between 200 nm and 280 nm.

13. The apparatus of claim 11, wherein the plurality of light emitting diodes are configured to generate a power flux of germicidal light sufficient to achieve a 2-log reduction bacterial contamination on surfaces within a room or area that are greater than 1 meter from the germicidal source within a given disinfection period.

14. The apparatus of claim 11, wherein the light source assembly is arranged above the support structure at an elevation between approximately 2 feet and approximately 4 feet from floor of a room in which the disinfection apparatus is arranged.

15. The apparatus of claim 11, further comprising:
one or more motion sensors and/or one or more occupancy sensors; and
circuitry for inhibiting or terminating activation of the plurality of light emitting diodes upon the one or more motions sensors detecting motion in a space in which the disinfection apparatus is arranged and/or the one or more occupancy sensors detecting occupancy in a space in which the disinfection apparatus is arranged.

16. A heat sink, comprising:
a fluid flow diverter having a shaft with a column portion and a tapered end portion, wherein a tip of the tapered end portion is arranged inward from an end of the heat sink by a distance less than approximately 15% of a length of the heat sink extending between its two opposing open ends, and wherein an interface between the column portion and the tapered end portion is arranged inward from the end of the heat sink by a distance that is between approximately 35% and approximately 45% of the length of the heat sink;
one or more walls encircling the fluid flow diverter;
a plurality of fins extending radially inward from interior surfaces of the one or more walls toward the fluid flow diverter; and
two open opposing ends at respectively opposing ends of the one or more walls.

17. The heat sink of claim 16, wherein at least some of the plurality of fins are coupled to the fluid flow diverter.

18. The heat sink of claim 16, wherein the fluid flow diverter comprises multiple support beams extending from one end of the shaft, and wherein the multiple support beams are coupled to at least some of the one or more walls.

19. The heat sink of claim 16, wherein the exterior surface area of the one or more walls is less than approximately 25 square inches.

20. The heat sink of claim 16, wherein the volume of the heat sink is less than approximately 200 cubic inches.
